(19) United States
(12) Patent Application Publication
(10) Pub. No.: US 2013/0211095 A1
(43) Pub. Date: Aug. 15, 2013

(54) NOVEL SYNTHESIS FOR THIAZOLIDINEDIONE COMPOUNDS

(71) Applicants: Timothy Parker, Portage, MI (US); Robert C. Gadwood, Portage, MI (US); Steven P. Tanis, Carlsbad, CA (US); Scott D. Larsen, South Lyon, MI (US); James R. Zeller, Scottsdale, AZ (US)

(72) Inventors: Timothy Parker, Portage, MI (US); Robert C. Gadwood, Portage, MI (US); Steven P. Tanis, Carlsbad, CA (US); Scott D. Larsen, South Lyon, MI (US); James R. Zeller, Scottsdale, AZ (US)

(73) Assignee: Metabolic Solutions Development Company, LLC, Kalamazoo, MI (US)

(21) Appl. No.: 13/641,898

(22) PCT Filed: Apr. 18, 2011
(86) PCT No.: PCT/US11/32816

§ 371 Date: Nov. 28, 2012

Related U.S. Application Data
(60) Provisional application No. 61/325,528, filed on Apr. 19, 2010.

(51) Int. Cl. C07D 277/04

(52) U.S. Cl. CPC C07D 277/04
USPC 546/314; 548/183; 548/110; 568/325

(57) ABSTRACT

The present invention provides novel methods for synthesizing PPARγ sparing compounds, e.g., thiazolidinediones, that are useful for preventing and/or treating metabolic disorders such as diabetes, obesity, hypertension, and inflammatory diseases.

### CROSS-REFERENCE TO RELATED APPLICATION

This PCT application claims the benefit of U.S. Application No. 61/325,528, which was filed on Apr. 19, 2010, and is hereby incorporated by reference in its entirety.

### TECHNICAL FIELD OF THE INVENTION

The present invention provides novel methods for synthesizing PPARγ sparing compounds, e.g., thiazolidinediones, that are useful for preventing and/or treating metabolic disorders such as diabetes, obesity, hypertension, and inflammatory diseases.

### BACKGROUND OF THE INVENTION

Over the past several decades, scientists have postulated that PPARγ is the generally accepted site of action for insulin sensitizing thiazolidinedione compounds.

Peroxisome Proliferator Activated Receptors (PPARs) are members of the nuclear hormone receptor super-family, which are ligand-activated transcription factors regulating gene expression. PPARs have been implicated in autoimmune diseases and other diseases, i.e., diabetes mellitus, cardiovascular and gastrointestinal disease, and Alzheimer's disease.

PPARγ is a key regulator of adipocyte differentiation and lipid metabolism. PPARγ is also found in other cell types including fibroblasts, myocytes, breast cells, human bone-marrow precursors, and macrophages/monocytes. In addition, PPARγ has been shown in macrophage foam cells in atherosclerotic plaques.

Thiazolidinediones, such as pioglitazone and rosiglitazone, developed originally for the treatment of type-2 diabetes, generally exhibit high affinity as PPARγ ligands. The finding that thiazolidinediones might mediate their therapeutic effects through direct interactions with PPARγ established the concept that PPARγ is a key regulator of glucose and lipid homeostasis. However, compounds that involve the activation of PPARγ, such as pioglitazone or rosiglitazone, also trigger sodium reabsorption and other unpleasant side effects that severely restrict their usage.

### SUMMARY OF THE INVENTION

In general, the invention relates to methods of synthesizing compounds that have reduced binding or activation of the nuclear transcription factor PPARγ when compared with high affinity PPARγ ligands such as rosiglitazone or pioglitazone. These novel methods are scalable for industrial production and employ safer, more stable, and/or less costly starting materials and process conditions.

Compounds exhibiting PPARγ activity induce transcription of genes that favor sodium reabsorption. Advantageously, the compounds produced by the synthetic methods of this invention have reduced binding or activation of the nuclear transcription factor PPARγ when compared with traditional high affinity PPARγ ligands (e.g., pioglitazone or rosiglitazone), and therefore produce fewer or diminished side effects (e.g., reduced augmentation of sodium reabsorption) that are associated with traditional high affinity PPARγ ligands, and are therefore more useful in treating hypertension, diabetes, and inflammatory diseases. Most specifically, the reduced PPARγ binding and reduced activity exhibited by these compounds, as compared with traditional high affinity PPARγ ligands (e.g., pioglitazone or rosiglitazone), are particularly useful for treating hypertension, diabetes, and inflammatory diseases both as single agents and in combination with other classes of antihypertensive agents because they lack the negative secondary effects of traditional PPARγ activating compounds. As hypertension and inflammatory diseases pose major risk factors in the onset of diabetes and pre-diabetes, these compounds are also useful for the treatment and prevention of diabetes and other inflammatory diseases. In fact, compounds synthesized by the present invention may induce remission of the symptoms of diabetes in a human patient.

One aspect of the present invention provides a novel synthesis for generating thiazolidinedione compounds that are useful for the treatment of metabolic disorders. Specifically, this synthesis is useful for preparing a compound of Formula I:

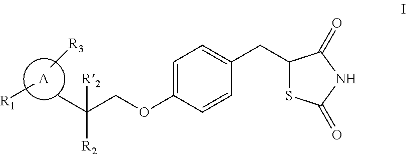

or a pharmaceutically acceptable salt thereof, wherein each of R₁ and R₃ is independently selected from H, halo, aliphatic, and alkoxy, wherein the aliphatic or alkoxy is optionally substituted with 1-3 of halo; each of R′₂ and R₂ are independently selected from —H, halo, hydroxy, or optionally substituted aliphatic, alkoxy, —O -acyl, —O-aroyl, —O-heteroaroyl, —O(SO₂)NH₂, —O—CH(Rₘ)OC(O)Rₙ, —O—CH(Rₘ)OP(O)(ORₙ)₂ —O—P(O)(ORₙ)₂, or

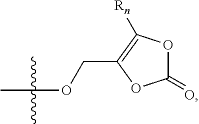

wherein each Rₘ is independently C₁₋₆ alkyl, each Rₙ is independently C₁₋₁₂ alkyl, C₃₋₈ cycloalkyl, or phenyl, each of which is optionally substituted; R₂ and R′₂ together form oxo, R₂ and R′₂ together form —O(CH₂)ₙO—, wherein n is 2 or 3, or R₂ and R′₂ together form —S(CH₂)ₘS—, wherein m is 2 or 3; and ring A is phenyl, pyridin-2-yl, pyridin-3-yl or pyridin-4-yl, each of which is substituted with R₁ and R₃ groups; comprising the step of reacting a compound of Formula 2A:

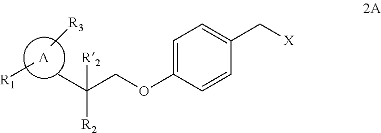

wherein X is a leaving group, with a compound of Formula 3A

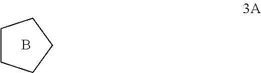

wherein ring B of Formula 3A is selected from

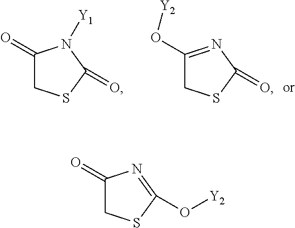

wherein Y₁ is hydrogen or PO_{N} and Y₂ is PG_{O}, wherein PG_{N} is a nitrogen protecting group and PG_{O} is an oxygen protecting group, to form a compound of Formula 4A; and

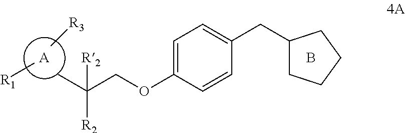

when Y₁ is other than hydrogen or when Y₂ is present, deprotecting the compound of Formula 4A to form a compound of Formula I.

In some implementations, X is a leaving group selected from —Br, —Cl, —I, —OMs, —OTs, —OTf, —OBs, —ONs, —O-tresylate, or —OPO(OR₄)₂, wherein each R₄ is independently C₁₋₄ alkyl or two of R₄ together with the oxygen and phosphorous atoms to which they are attached form a 5-7 membered ring.

Other implementations further comprise modifying a compound of Formula 5A

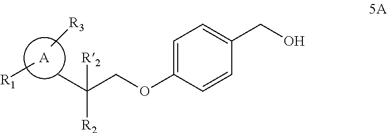

to form a compound of Formula 2A.

Some implementations further comprise reacting a compound of Formula 6A

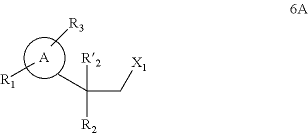

wherein X₁ is halo, with a compound having the structure

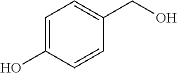

under basic conditions to form a compound of Formula 5A.

In some implementations, the compound of Formula 6A comprises

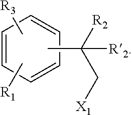

For example, the compound of Formula 6A comprises

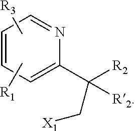

In other examples, the compound of Formula 6A comprises

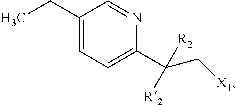

wherein X₁ is —Br or —Cl. And in some of these examples, R₂ and R′₂ together form oxo.

In other implementations, the compound of Formula 6A comprises

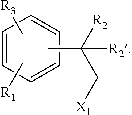

For example, the compound of Formula 6A comprises

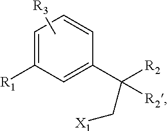

wherein R₁ is a C₁₋₆ alkyl or C₁₋₆ alkoxy, either of which is optionally substituted with 1-3 halo. In other examples, the compound of Formula 6A comprises

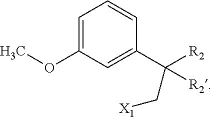

And, in some of these examples, R₂ and R′₂ together form oxo.

In alternative implementations, X and X₁ are independently selected from —Br and —Cl.

And, in some implementations, ring B of Formula 3A is

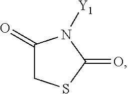

Y₁ is PG_{N}, and PO_{N} is a nitrogen protecting group selected from Cbz, Moz, Boc, Fmoc, Ac, Bz, Bn, PMB, DMPM, PMP, or trityl.

In other implementations, ring B of Formula 3A is

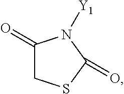

and Y₁ is hydrogen.

In some implementations, ring B of Formula 3A is

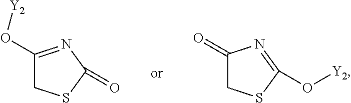

Y₂ is PG_{O}, and PG_{O} is an oxygen protecting group selected from —Si(R₆)₃, optionally substituted alkyl, or optionally substituted alkylcarbonyl, wherein each R₆ is independently straight or branched C₁₋₄ alkyl. For example, ring B of Formula 3A is

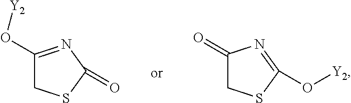

Y₂ is PG_{O}, and PG_{O} is —Si(R₆)_{3,} wherein each R₆ is independently selected from methyl, ethyl, propyl, isopropyl, butyl, sec-butyl or tert-butyl. In other examples, ring B of Formula 3A is

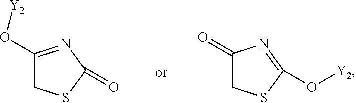

Y₂ is PG_{O}, and PG_{O} is a C₁₋₆ alkyl or a C₁₋₆ alkylcarbonyl.

Another aspect of the present invention provides a compound selected from

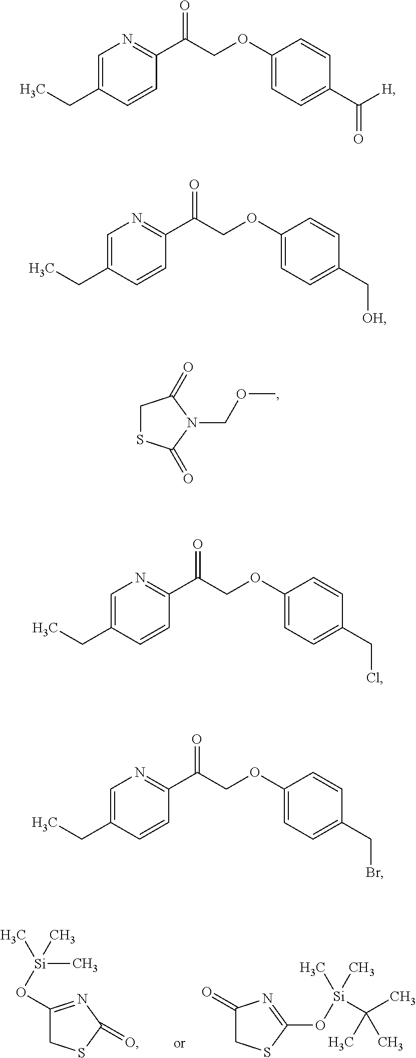

And, another aspect of the present invention provides a compound selected from

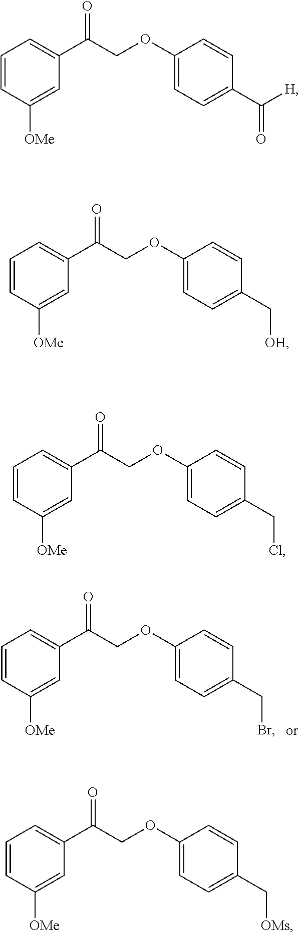

### DETAILED DESCRIPTION

The present invention provides novel methods for preparing thiazolidinedione compounds having reduced PPARγ activity and/or binding.

As used herein, the following definitions shall apply unless otherwise indicated.

### I. DEFINITIONS

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M.B. and March, J., John Wiley & Sons, New York: 2001, the entire contents of which are hereby incorporated by reference.

As described herein, “protecting group” refers to a moiety or functionality that is introduced into a molecule by chemical modification of a functional group in order to obtain chemoselectivity in a subsequent chemical reaction. Standard protecting groups are provided in Greene and Wuts : “Greene's Protective Groups in Organic Synthesis” 4th Ed, Wuts, P.G.M. and Greene, T.W., Wiley-Interscience, New York:2006.

As described herein, compounds of the invention may optionally be substituted with one or more substituents, such as those illustrated generally above, or as exemplified by particular classes, subclasses, and species of the invention.

As used herein, the term “hydroxyl” or “hydroxy” refers to an —OH moiety.

As used herein the term “aliphatic” encompasses the terms alkyl, alkenyl, alkynyl, each of which being optionally substituted as set forth below.

As used herein, an “alkyl” group refers to a saturated aliphatic hydrocarbon group containing 1-12 (e.g., 1-8, 1-6, or 14) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo, phospho, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, cycloaliphaticamino, or heterocycloaliphaticamino], sulfonyl [e.g., aliphatic-SO₂—], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, aralkyl, (alkoxyaryl)alkyl, (sulfonylamino)alkyl (such as (alkyl-SO₂-amino)alkyl), aminoalkyl, amidoalkyl, (cycloaliphatic)alkyl, or haloalkyl.

As used herein, an “alkenyl” group refers to an aliphatic carbon group that contains 2-8 (e.g., 2-12, 2-6, or 2-4) carbon atoms and at least one double bond. Like an alkyl group, an alkenyl group can be straight or branched. Examples of an alkenyl group include, but are not limited to allyl, isoprenyl, 2-butenyl, and 2-hexenyl. An alkenyl group can be optionally substituted with one or more substituents such as halo, phospho, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, cycloaliphaticamino, heterocycloaliphaticamino, or aliphaticsulfonylamino], sulfonyl [e.g., alkyl-SO₂—, cycloaliphatic-SO₂—, or aryl-SO₂—], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroaralkoxy, alkoxycarbonyl, alkylcarbonyloxy, or hydroxy. Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-SO₂-amino)alkenyl), aminoalkenyl, amidoalkenyl, (cycloaliphatic)alkenyl, or haloalkenyl.

As used herein, an “alkynyl” group refers to an aliphatic carbon group that contains 2-8 (e.g., 2-12, 2-6, or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as aroyl, heteroaroyl, alkoxy, cycloalkyloxy, heterocycloalkyloxy, aryloxy, heteroaryloxy, aralkyloxy, nitro, carboxy, cyano, halo, hydroxy, sulfo, mercapto, sulfanyl [e.g., aliphaticsulfanyl or cycloaliphaticsulfanyl], sulfinyl [e.g., aliphaticsulfinyl or cycloaliphaticsulfinyl], sulfonyl [e.g., aliphatic-SO₂-, aliphaticamino-SO₂—, or cycloaliphatic-SO₂—], amido [e.g., aminocarbonyl, alkylaminocarbonyl, alkylcarbonylamino, cycloalkylaminocarbonyl, heterocycloalkylaminocarbonyl, cycloalkylcarbonylamino, arylaminocarbonyl, arylcarbonylamino, aralkylcarbonylamino, (heterocycloalkyl)carbonylamino, (cycloalkylalkyl)carbonylamino, heteroaralkylcarbonylamino, heteroarylcarbonylamino or heteroarylaminocarbonyl], urea, thiourea, sulfamoyl, sulfamide, alkoxycarbonyl, alkylcarbonyloxy, cycloaliphatic, heterocycloaliphatic, aryl, heteroaryl, acyl [e.g., (cycloaliphatic)carbonyl or (heterocycloaliphatic)carbonyl], amino [e.g., aliphaticamino], sulfoxy, oxo, carboxy, carbamoyl, (cycloaliphatic)oxy, (heterocycloaliphatic)oxy, or (heteroaryl)alkoxy.

As used herein, an “amido” encompasses both “aminocarbonyl” and “carbonylamino”. These terms when used alone or in connection with another group refer to an amido group such as —N(R^{X})—C(O)—R^{V} or —C(O)—N(R^{X})₂, when used terminally, and —C(O)—N(R^{X})— or —N(R^{X})—C(O)— when used internally, wherein R^{X} and R^{Y} can be, independently, aliphatic, cycloaliphatic, aryl, araliphatic, heterocycloaliphatic, heteroaryl or heteroaraliphatic. Examples of amido groups include alkylamido (such as alkylcarbonylamino or alkylaminocarbonyl), (heterocycloaliphatic)amido, (heteroaralkyl)amido, (heteroaryl)amido, (heterocycloalkyl)alkylamido, arylamido, aralkylamido, (cycloalkyl)alkylamido, or cycloalkylamido.

As used herein, an “amino” group refers to —NR^{X}R^{Y} wherein each of R^{X} and R^{Y} is independently hydrogen, aliphatic, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, or arylamino. When the term “amino” is not the terminal group (e.g., alkylcarbonylamino), it is represented by —NR^{X}—, where R^{X} has the same meaning as defined above.

As used herein, an “aryl” group used alone or as part of a larger moiety as in “aralkyl”, “aralkoxy”, or “aryloxyalkyl” refers to monocyclic (e.g., phenyl); bicyclic (e.g., indenyl, naphthalenyl, tetrahydronaphthyl, tetrahydroindenyl); and tricyclic (e.g., fluorenyl tetrahydrofluorenyl, or tetrahydroanthracenyl, anthracenyl) ring systems in which the monocyclic ring system is aromatic or at least one of the rings in a bicyclic or tricyclic ring system is aromatic. The bicyclic and tricyclic groups include benzofused 2-3 membered carbocyclic rings. For example, a benzofused group includes phenyl fused with two or more C₄₋₈ carbocyclic moieties. An aryl is optionally substituted with one or more substituents including aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); nitro; carboxy; amido; acyl [e.g., (aliphatic)carbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl]; sulfonyl [e.g., aliphatic-SO₂— or amino-SO₂—]; sulfinyl [e.g., aliphatic-S(O)— or cycloaliphatic-S(O)—]; sulfanyl [e.g., aliphatic-S—]; cyano; halo; hydroxy; mercapto; sulfoxy; urea; thiourea; sulfamoyl; sulfamide; or carbamoyl. Alternatively, an aryl can be unsubstituted.

Non-limiting examples of substituted aryls include haloaryl [e.g., mono-, di (such as p,m-dihaloaryl), and (trihalo)aryl]; (carboxy)aryl [e.g., (alkoxycarbonyl)aryl, ((aralkyl)carbonyloxy)aryl, and (alkoxycarbonyl)aryl]; (amido)aryl [e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl]; aminoaryl [e.g., ((alkylsulfonyl)amino)aryl or ((dialkyl)amino)aryl]; (cyanoalkyl)aryl; (alkoxy)aryl; (sulfamoyl)aryl [e.g., (aminosulfonyparyl]; (alkylsulfonyl)aryl; (cyano)aryl; (hydroxyalkyl)aryl; ((alkoxy)alkyl)aryl; (hydroxy)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; or (m-(heterocycloaliphatic)-o-(alkyl))aryl.

As used herein, an “araliphatic” such as an “aralkyl” group refers to an aliphatic group (e.g., a C₁₋₄ alkyl group) that is substituted with an aryl group. “Aliphatic,” “alkyl,” and “aryl” are defined herein. An example of an araliphatic such as an aralkyl group is benzyl.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl. An aralkyl is optionally substituted with one or more substituents such as aliphatic [e.g., alkyl, alkenyl, or alkynyl, including carboxyalkyl, hydroxyalkyl, or haloalkyl such as trifluoromethyl], cycloaliphatic [e.g., cycloalkyl or cycloalkenyl], (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, amido [e.g., aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, or heteroaralkylcarbonylamino], cyano, halo, hydroxy, acyl, mercapto, alkylsulfanyl, sulfoxy, urea, thiourea, sulfamoyl, sulfamide, oxo, or carbamoyl.

As used herein, a “bicyclic ring system” includes 6-12 (e.g., 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls. Furthermore, bicyclic ring systems include bridged bicyclic moieties and fused bicyclic moieties.

As used herein, a “cycloaliphatic” group encompasses a “cycloalkyl” group and a “cycloalkenyl” group, each of which being optionally substituted as set forth below.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, or ((aminocarbonyl)cycloalkyl)cycloalkyl.

A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, cyclopentenyl, bicyclo[2.2.2]octenyl, or bicyclo[3.3.1]nonenyl.

A cycloalkyl or cycloalkenyl group can be optionally substituted with one or more substituents such as phospho, aliphatic [e.g., alkyl, alkenyl, or alkynyl], cycloaliphatic, (cycloaliphatic) aliphatic, heterocycloaliphatic, (heterocycloaliphatic) aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino], nitro, carboxy [e.g., HOOC-, alkoxycarbonyl, or alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl], cyano, halo, hydroxy, mercapto, sulfonyl [e.g., alkyl-SO₂— and aryl-SO₂—], sulfinyl [e.g., alkyl-S(O)—], sulfanyl [e.g., alkyl-S—], sulfoxy, urea, thiourea, sulfamoyl, sulfamide, oxo, or carbamoyl.

As used herein, the term “heterocycloaliphatic” encompasses heterocycloalkyl groups and heterocycloalkenyl groups, each of which being optionally substituted as set forth below.

As used herein, a “heterocycloalkyl” group refers to a 3-10 membered mono- or bicylic (fused or bridged) (e.g., 5- to 10-membered mono- or bicyclic) saturated ring structure, in which one or more of the ring atoms is a heteroatom (e.g., N, O, S, or combinations thereof). Examples of a heterocycloalkyl group include azetidinyl, piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.0^{3,7}]nonyl. A monocyclic heterocycloalkyl group can be fused with a phenyl moiety to form structures, such as tetrahydroisoquinoline, which would be categorized as heteroaryls.

A “heterocycloalkenyl” group, as used herein, refers to a mono- or bicylic (e.g., 5- to 10-membered mono- or bicyclic) non-aromatic ring structure having one or more double bonds, and wherein one or more of the ring atoms is a heteroatom (e.g., N, O, or S). Monocyclic and bicyclic heterocycloaliphatics are numbered according to standard chemical nomenclature.

A heterocycloalkyl or heterocycloalkenyl group can be optionally substituted with one or more substituents such as phospho, aliphatic [e.g., alkyl, alkenyl, or alkynyl], cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic) aliphatic)carbonylamino, (aryl)carbonylamino, (araliphatic)carbonylamino, (heterocycloaliphatic)carbonylamino, ((heterocycloaliphatic) aliphatic)carbonylamino, (heteroaryl)carbonylamino, or (heteroaraliphatic)carbonylamino], nitro, carboxy [e.g., HOOC-, alkoxycarbonyl, or alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl], nitro, cyano, halo, hydroxy, mercapto, sulfonyl [e.g., alkylsulfonyl or arylsulfonyl], sulfinyl [e.g., alkylsulfinyl], sulfanyl [e.g., alkylsulfanyl], sulfoxy, urea, thiourea, sulfamoyl, sulfamide, oxo, or carbamoyl.

A “heteroaryl” group, as used herein, refers to a monocyclic, bicyclic, or tricyclic ring system having 4 to 15 ring atoms wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having 2 to 3 rings. For example, a benzofused group includes benzo fused with one or two 4 to 8 membered heterocycloaliphatic moieties (e.g., indolizyl, indolyl, isoindolyl, 3H-indolyl, indolinyl, benzo[b]furyl, benzo[b]thiophenyl, quinolinyl, or isoquinolinyl). Some examples of heteroaryl are pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl,cinnolyl, phthalazyl, quinazolyl, quinoxalyl, isoquinolyl, 4H-quinolizyl, benzo-1,2,5-thiadiazolyl, or 1,8-naphthyridyl.

Without limitation, monocyclic heteroaryls include furyl, thiophenyl, 2H-pyrrolyl, pyrrolyl, oxazolyl, thazolyl, imidazolyl, pyrazolyl, isoxazolyl, isothiazolyl, 1,3,4-thiadiazolyl, 2H-pyranyl, 4-H-pranyl, pyridyl, pyridazyl, pyrimidyl, pyrazolyl, pyrazyl, or 1,3,5-triazyl. Monocyclic heteroaryls are numbered according to standard chemical nomenclature.

Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indolizyl, isoindolyl, indolyl, benzo[b]furyl, bexo[b]thiophenyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); carboxy; amido; acyl [ e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl]; sulfonyl [e.g., aliphaticsulfonyl or aminosulfonyl]; sulfinyl [e.g., aliphaticsulfinyl]; sulfanyl [e.g., aliphaticsulfanyl]; nitro; cyano; halo; hydroxy; mercapto; sulfoxy; urea; thiourea; sulfamoyl; sulfamide; or carbamoyl. Alternatively, a heteroaryl can be unsubstituted.

Non-limiting examples of substituted heteroaryls include (halo)heteroaryl [e.g., mono- and di-(halo)heteroaryl]; (carboxy)heteroaryl [e.g., (alkoxycarbonypheteroaryl]; cyanoheteroaryl; aminoheteroaryl [e.g., ((alkylsulfonyl)amino)heteroaryl and ((dialkyl)amino)heteroaryl]; (amido)heteroaryl [e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyl)amino)heteroaryl]; (cyanoalkyl)heteroaryl; (alkoxy)heteroaryl; (sulfamoyl)heteroaryl [e.g., (aminosulfonyl)heteroaryl]; (sulfonyl)heteroaryl [e.g., (alkylsulfonyl)heteroaryl]; (hydroxyalkyl)heteroaryl; (alkoxyalkyl)heteroaryl; (hydroxy)heteroaryl; ((carboxy)alkyl)heteroaryl; (((dialkyl)amino)alkyl]heteroaryl; (heterocycloaliphatic)heteroaryl; (cycloaliphatic)heteroaryl; (nitroalkyl)heteroaryl; (((alkylsulfonyl)amino)alkyl)heteroaryl; ((alkylsulfonyl)alkyl)heteroaryl; (cyanoalkyl)heteroaryl; (acyl)heteroaryl [e.g., (alkylcarbonyl)heteroaryl]; (alkyl)heteroaryl; or (haloalkyl)heteroaryl [e.g., trihaloalkylheteroaryl].

A “heteroaraliphatic (such as a heteroaralkyl group) as used herein, refers to an aliphatic group (e.g., a C₁₋₄ alkyl group) that is substituted with a heteroaryl group. “Aliphatic,” “alkyl,” and “heteroaryl” have been defined above.

A “heteroaralkyl” group, as used herein, refers to an alkyl group (e.g., a C₁₋₄ alkyl group) that is substituted with a heteroaryl group. Both “alkyl” and “heteroaryl” have been defined above. A heteroaralkyl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, “cyclic moiety” and “cyclic group” refer to mono-, bi-, and tri-cyclic ring systems including cycloaliphatic, heterocycloaliphatic, aryl, or heteroaryl, each of which has been previously defined.

As used herein, a “bridged bicyclic ring system” refers to a bicyclic heterocyclicalipahtic ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.3.2]decyl, 2-oxabicyclo[2.2.2]octyl, 1-azabicyclo[2.2.2]octyl, 3-azabicyclo[3.2.1]octyl, and 2,6-dioxa-tricyclo[3.3.1.0^{3a}]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, an “acyl” group refers to a formyl group or R^{X}—C(O)— (such as alkyl-C(O)—, also referred to as “alkylcarbonyl”) where R^{X} and “alkyl” have been defined previously. Acetyl and pivaloyl are examples of acyl groups.

As used herein, an “aroyl” or “heteroaroyl” refers to an aryl-C(O)— or a heteroaryl-C(O)—. The aryl and heteroaryl portion of the aroyl or heteroaroyl is optionally substituted as previously defined.

As used herein, an “alkoxy” group refers to an alkyl-O— group where “alkyl” has been defined previously.

As used herein, a “carbamoyl” group refers to a group having the structure —O—CO—NR^{X}R^{Y} or —NR^{X}—O—R^{Z}, wherein R^{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, —COOR^{X}, —OC(O)H, —OC(O)R^{X}, when used as a terminal group; or —OC(O)— or —C(O)O— when used as an internal group.

As used herein, a “haloaliphatic” group refers to an aliphatic group substituted with 1-3 halogen. For instance, the term haloalkyl includes the group —CF₃.

As used herein, a “mercapto” group refers to —SH.

As used herein, a “sulfo” group refers to —SO₃H or —SO₃R^{X} when used terminally or —S(O)₃— when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfamoyl” group refers to the structure —O—S(O)₂—NR^{Y}R^{Z} wherein R^{Y} and R^{Z} have been defined above.

As used herein, a “sulfonamide” group refers to the structure —S(O)₂—NR^{X}R^{Y} or —NR^{X}—S(O)₂—R^{Z} when used terminally; or —S(O)₂—NR^{X}— or —NO^{X}—S(O)₂— when used internally, wherein R^{X}, R^{Y}, and R^{Z} are defined above.

As used herein a “sulfanyl” group refers to —S—R^{X} when used terminally and —S— when used internally, wherein R^{X} has been defined above. Examples of sulfanyls include aliphatic-S-, cycloaliphatic-S—, aryl-S-, or the like.

As used herein a “sulfinyl” group refers to —S(O)—R^{X} when used terminally and —S(O)— when used internally, wherein R^{X} has been defined above. Exemplary sulfinyl groups include aliphatic-S(O)—, aryl-S(O)—, (cycloaliphatic(aliphatic))-S(O)—, cycloalkyl-S(O)—, heterocycloaliphatic-S(O)—, heteroaryl-S(O)—, or the like.

As used herein, a “sulfonyl” group refers to-S(O)₂—R^{X} when used terminally and —S(O)₂— when used internally, wherein R^{X} has been defined above. Exemplary sulfonyl groups include aliphatic-S(O)₂—, aryl-S(O)₂—, (cycloaliphatic(aliphatic))-S(O)₂—, cycloaliphatic-S(O)₂—, heterocycloaliphatic-S(O)₂—, heteroaryl-S(O)₂‘3, (cycloaliphatic(amido(aliphatic)))-S(O)₂—, or the like.

As used herein, a “sulfoxy” group refers to —O—SO—R^{X} or —SO—O—R^{X}, when used terminally and —O—S(O)— or —S(O)—O— when used internally, where R^{X} has been defined above.

As used herein, a “halogen” or “halo” group refers to fluorine, chlorine, bromine or iodine.

As used herein, an “alkoxycarbonyl,” which is encompassed by the term carboxy, used alone or in connection with another group refers to a group such as alkyl-O—C(O)—.

As used herein, an “alkoxyalkyl” refers to an alkyl group such as alkyl-O-alkyl-, wherein alkyl has been defined above.

As used herein, a “carbonyl” refer to —C(O)—.

As used herein, an “oxo” refers to ═O.

As used herein, the term “phospho” refers to phosphinates and phosphonates. Examples of phosphinates and phosphonates include —P(O)(R^{P})₂, wherein R^{P} is aliphatic, alkoxy, aryloxy, heteroaryloxy, (cycloaliphatic)oxy, (heterocycloaliphatic)oxy aryl, heteroaryl, cycloaliphatic or amino.

As used herein, an “aminoalkyl” refers to the structure (R^{X})₂N-alkyl-.

As used herein, a “cyanoalkyl” refers to the structure (NC)-alkyl-.

As used herein, a “urea” group refers to the structure —NR^{X}—CO—NR^{Y}R^{Z} and a “thiourea” group refers to the structure —NR^{X}—CS—NR^{Y}R^{Z} when used terminally and —NR^{X}—CO—NR^{Y}— or —NR^{X}—CS—NR^{Y}— when used internally, wherein le, R^{X}, and R^{Z} have been defined above.

As used herein, a “guanidine” group refers to the structure —N═C(N(R^{X}R^{Y}))N(R^{X}R^{Y}) or —NR^{X}—C(═NR^{X})NR^{X}R^{Y} it wherein R^{X} and R^{Y} have been defined above.

As used herein, the term “amidino” group refers to the structure —C═(NR^{X})N(R^{X}R^{Y}) wherein R^{X} and le have been defined above.

In general, the term “vicinal” refers to the placement of substituents on a group that includes two or more carbon atoms, wherein the substituents are attached to adjacent carbon atoms.

In general, the term “geminal” refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl is an example of a carboxy group used terminally. A group is internal when the group is present in the middle of a substituent of the chemical structure. Alkylcarboxy (e.g., alkyl-C(O)O— or alkyl-OC(O)—) and alkylcarboxyaryl (e.g., alkyl-C(O)O-aryl- or alkyl-O(CO)-aryl-) are examples of carboxy groups used internally.

As used herein, an “aliphatic chain” refers to a branched or straight aliphatic group (e.g., alkyl groups, alkenyl groups, or alkynyl groups). A straight aliphatic chain has the structure —[CH₂]_{Y}—, where v is 1-12. A branched aliphatic chain is a straight aliphatic chain that is substituted with one or more aliphatic groups. A branched aliphatic chain has the structure —[CQQ]_{Y}— where Q is independently a hydrogen or an aliphatic group; however, Q shall be an aliphatic group in at least one instance. The term aliphatic chain includes alkyl chains, alkenyl chains, and alkynyl chains, where alkyl, alkenyl, and alkynyl are defined above.

The phrase “optionally substituted” is used interchangeably with the phrase “substituted or unsubstituted.” As described herein, compounds of the invention can optionally be substituted with one or more substituents, such as are illustrated generally above, or as exemplified by particular classes, subclasses, and species of the invention. As described herein, the variables R₁, R₂, R′₂, R₃, R₄, and other variables contained in formulae described herein encompass specific groups, such as alkyl and aryl. Unless otherwise noted, each of the specific groups for the variables R₁, R₂, R′₂, R₃, R₄, and other variables contained therein can be optionally substituted with one or more substituents described herein. Each substituent of a specific group is further optionally substituted with one to three of halo, cyano, oxo, alkoxy, hydroxy, amino, nitro, aryl, cycloaliphatic, heterocycloaliphatic, heteroaryl, haloalkyl, and alkyl. For instance, an alkyl group can be substituted with alkylsulfanyl and the alkylsulfanyl can be optionally substituted with one to three of halo, cyano, oxo, alkoxy, hydroxy, amino, nitro, aryl, haloalkyl, and alkyl. As an additional example, the cycloalkyl portion of a (cycloalkyl)carbonylamino can be optionally substituted with one to three of halo, cyano, alkoxy, hydroxy, nitro, haloalkyl, and alkyl. When two alkoxy groups are bound to the same atom or adjacent atoms, the two alkoxy groups can form a ring together with the atom(s) to which they are bound.

In general, the term “substituted,” whether preceded by the term “optionally” or not, refers to the replacement of one or more hydrogen atoms in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this invention are those combinations that result in the formation of stable or chemically feasible compounds.

The phrase “stable or chemically feasible,” as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

As used herein, an “effective amount” is defined as the amount required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, New York, 537 (1970). As used herein, “patient” refers to a mammal, including a human.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays, or as therapeutic agents.

Chemical structures and nomenclature are derived from ChemDraw, version 11.0.1, Cambridge, Mass.

### II. COMMONLY USED ABBREVIATIONS

The following abbreviations are used:

PG protecting group

LG leaving group

DCM dichloromethane

Ac acetyl

DMF dimethylformamide

EtOAc ethyl acetate

DMSO dimethyl sulfoxide

MeCN acetonitrile

TCA trichloroacetic acid

ATP adenosine triphosphate

EtOH ethanol

Ph phenyl

Me methyl

Et ethyl

Bu butyl

DEAD diethylazodicarboxylate

HEPES 4-(2-hydroxyethyl)-1-piperazineethanesulfonic acid

BSA bovine serum albumin

DTT dithiothreitol

MOPS 4-morpholinepropanesulfonic acid

NMR nuclear magnetic resonance

HPLC high performance liquid chromatography

LCMS liquid chromatography-mass spectrometry

TLC thin layer chromatography

Rt retention time

HOBt hydroxybenzotriazole

Ms mesyl

Ts tosyl

Tf triflyl

Bs besyl

Ns nosyl

Cbz carboxybenzyl

Moz p-methoxybenzyl carbonyl

Boc tert-butyloxycarbonyl

Fmoc 9-fluorenylmethyloxycarbonyl

Bz benzoly

Bn benzyl

PMB p-methoxybenzyl

DMPM 3,4-dimethoxybenzyl

PMP p-methoxyphenyl

### III. METHODS OF SYNTHESIZING COMPOUNDS OF FORMULA

One aspect of the present invention provides a novel synthesis for generating thiazolidinedione compounds that are useful for the treatment of metabolic disorders. One aspect of the present invention provides a novel synthesis for generating thiazolidinedione compounds that are useful for the treatment of metabolic disorders. This synthesis is useful for preparing a compound of Formula I:

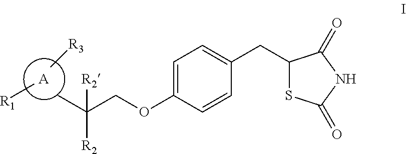

or a pharmaceutically acceptable salt thereof, wherein each of R₁ and R₃ is independently selected from H, halo, aliphatic, and alkoxy, wherein the aliphatic or alkoxy is optionally substituted with 1-3 of halo; each of R′₂ and R₂ are independently selected from —H, halo, hydroxy, or optionally substituted aliphatic, alkoxy, —O-acyl, —O-aroyl, -O-heteroaroyl, —O(SO₂)NH₂, —O—CH(Rₘ)OC(O)Rₙ, —O—CH(Rₘ)OP(O)(ORₙ)₂ —O—P(O)(ORₙ)₂, or

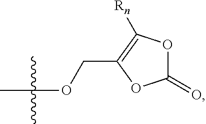

wherein each Rₘ is independently C₁₋₆ alkyl, each Rₙ is independently C₁₋₁₂ alkyl, C₃₋₈ cycloalkyl, or phenyl, each of which is optionally substituted; R₂ and R′₂ together form oxo, R₂ and R′₂ together form —O(CH₂)ₙO—, wherein n is 2 or 3, or R₂ and R′₂ together form —S(CH₂)ₘS—, wherein m is 2 or 3; and ring A is phenyl, pyridin-2-yl, pyridin-3-yl or pyridin-4-yl, each of which is substituted with R₁ and R₃ groups; comprising the step of reacting a compound of Formula 2A

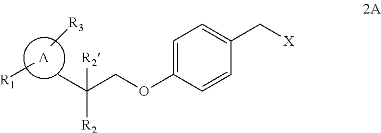

wherein X is a leaving group, with a compound of Formula 3A

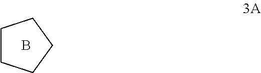

wherein ring B of Formula 3A is selected from

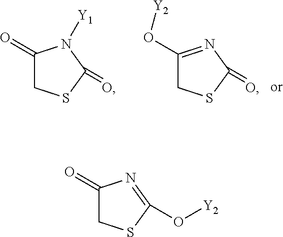

wherein Y₁ is hydrogen or PG_{N} and Y₂ is PG_{O}, wherein PG_{N} is a nitrogen protecting group and PG_{O} is an oxygen protecting group, to form a compound of Formula 4A; and

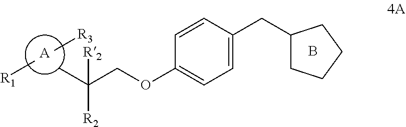

when Y₁ is other than hydrogen, i.e., Y₁ is PG_{N}, or when Y₂ is present, deprotecting the compound of Formula 4A to form a compound of Formula I.

It is noted that when ring B of Formula 3A is

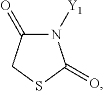

and Y₁ is other than hydrogen, i.e., Y₁ is PG_{N}, the nitrogen atom is considered to be protected, i.e., not of the form

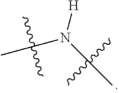 [SMILES: *N*]

Also, when ring B of Formula 3A is

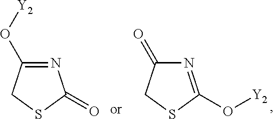

and Y₂ is present, the oxygen atom is considered to be protected. In either case where the nitrogen atom or the oxygen atom is protected, the compound of Formula 4A must undergo an additional deprotection step (e.g., treatment with a reagent (e.g., an aqueous acid or an aqueous base)) to form a compound of Formula I. However, when Y₁ is hydrogen on ring B, then the compound of Formula 4A is a compound of Formula I.

In several implementations, X is a leaving group selected from —Br, —Cl, —I, —OMs, —OTs, —OTf, —OBs, —ONs, —O-tresylate, or —OPO(OR₄)₂, wherein each R₄ is independently C₁₋₄ alkyl or two of R₄ together with the oxygen and phosphorous atoms to which they are attached form a 5-7 membered ring. For example, X is a halo. In other examples, X is —Cl, —Br, or —I.

Some implementations comprise modifying a compound of Formula 5A

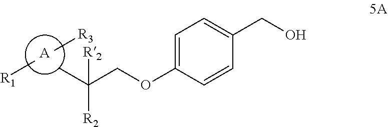

to form a compound of Formula 2A. In several examples, this modification includes modifying the hydroxy functionality into a leaving group or substituting a leaving group for the hydroxy functionality in the compound of Formula 5A.

Other implementations comprise reacting a compound of Formula 6A

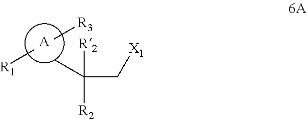

wherein X₁ is halo, with a compound having the structure

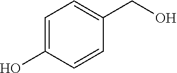

under basic conditions to form a compound of Formula 5A. For example, the compound of Formula 6A is reacted with a compound having the structure

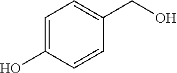

in a solvent system comprising K₂CO₃ and acetone.

In some implementations, the compound of Formula 6A comprises

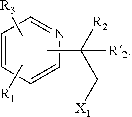

For example, the compound of Formula 6A comprises

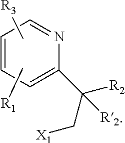

In other examples, the compound of Formula 6A comprises

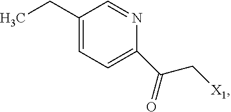

wherein X₁ is —Br or —Cl.

In other implementations, the compound of Formula 6A comprises

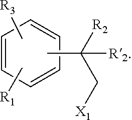

For example, the compound of Formula 6A comprises

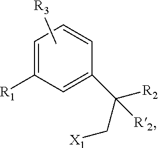

wherein R₁ is a C₁₋₆ alkyl or C₁₋₆ alkoxy, either of which is optionally substituted with 1-3 halo. In some examples, the compound of Formula 6A comprises

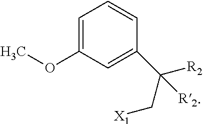

And in some instances, R₂ and R′₂, of the compound of Formula 6A together form oxo.

In other instances, X and X₁ are independently selected from —Br and —Cl.

In some implementations, ring B of Formula 3A is

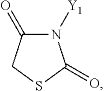

Y₁ is PO_{N}, and PU_{N} is a nitrogen protecting group selected from Cbz, Moz, Boc, Fmoc, Ac, Bz, Bn, PMB, DMPM, PMP, or trityl. In others, ring B of Formula 3A is

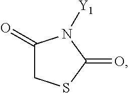

and Y₁ is hydrogen.

In some implementations, ring B of Formula 3A is

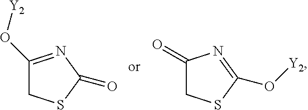

Y₂ is PG_{O}, and PG_{O} is an oxygen protecting group selected from —Si(R₆)₃, optionally substituted alkyl, or optionally substituted alkylcarbonyl, wherein each R₆ is independently straight or branched C₁₋₄ alkyl or phenyl. For example, ring B of Formula 3A is

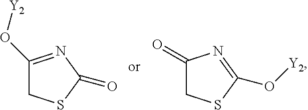

Y₂ is PG_{O}, and PG_{O} is —Si(R₆)₃, wherein each R₆ is independently selected from methyl, ethyl, propyl, isopropyl, butyl, sec-butyl, tert-butyl or phenyl. In other examples, ring B of Formula 3A is

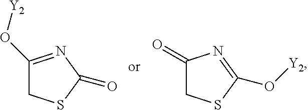

Y₂ is PG_{O}, and PG_{O} is a C₁₋₆ alkyl or a C₁₋₆ alkylcarbonyl.

In several implementations, R′₂ and R₂, in any of the formulae above, are independently selected from —OMe, —OEt or other optionally substituted O—C₁₋₆ alkyl groups. In other implementations, R′₂ and R₂ are groups that can readily be converted to oxo without performing an oxidation reaction.

In some implementations, X is a leaving group that allows for nucleophilic displacement by 1,3-thiazolidine-2,4-dione or protected 1,3-thiazolidine-2,4-dione. For example, X is —Br, —Cl, —I, —OMs, —OTs, —ONs, or —OPO(OR₄)₂, wherein each R₄ is independently C₁₋₁₂ alkyl, C₃₋₈ cycloalkyl, or phenyl, each of which is optionally substituted.

In some implementations where Y₁ is PG_{N}, in any of the formulae above, PG_{N} is Ac, methoxymethyl, ethoxyethyl, ethoxymethyl, p-methoxybenxyl, methoxycarbonyl, ethoxycarbonyl, or triphenylmethyl.

### IV. EXEMPLARY SYNTHESES

The following synthetic schemes represent example implementations of the present invention.

Scheme 1 illustrates one exemplary alkylation of a compound of Formula 3A.

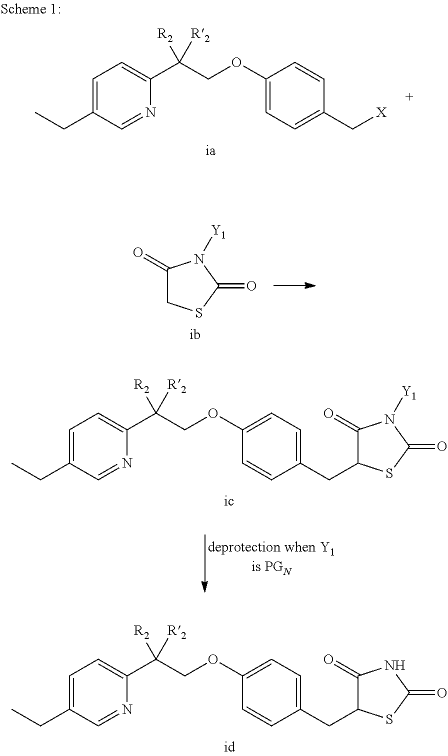

wherein R₂, R′₂, X, Y₁, and PG_{N} are defined above.

In several implementations, starting material is is generated according to Scheme 1A, below:

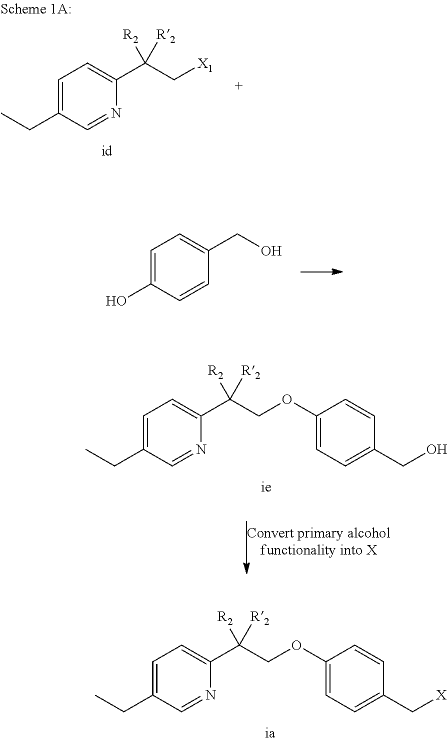

wherein R₂, R′₂, X₁, and X are defined above.

Scheme 2 illustrates another exemplary alkylation of a compound of Formula 3A.

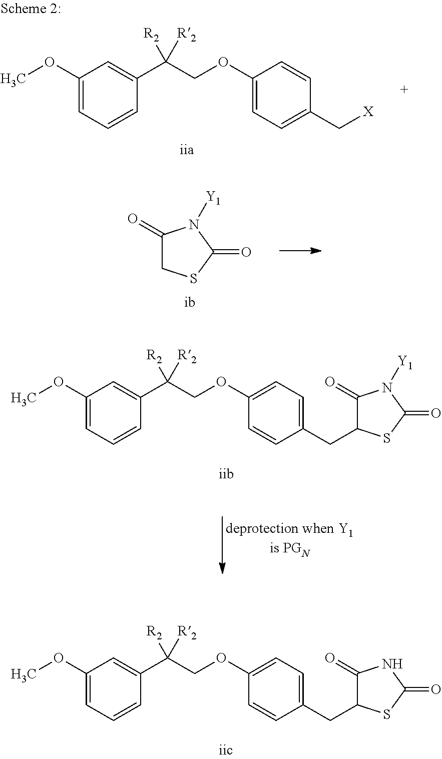

wherein R₂, R′₂, and X are defined in Formula I, above.

In some implementations, starting material iia is generated according to Scheme 2A, below:

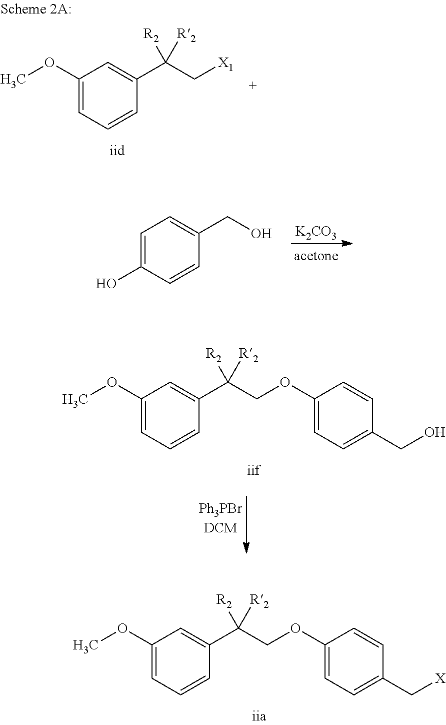

wherein X is —Br, and R₂, R′₂, and X₁ are defined above.

### V. NOVEL COMPOUNDS

Another aspect of the present invention provides novel compounds that are useful in the synthesis of compounds of Formula I. For example, one aspect of the present invention provides a compound selected from

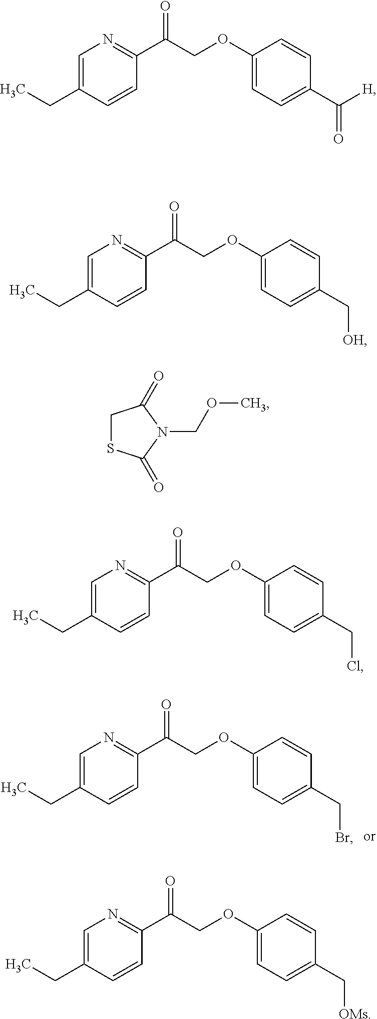

Another aspect of the present invention provides a compound of Formula II

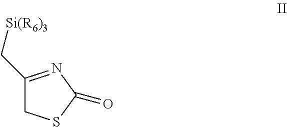

wherein R₆ is defined above.

In some implementations, the compound of Formula II is selected from

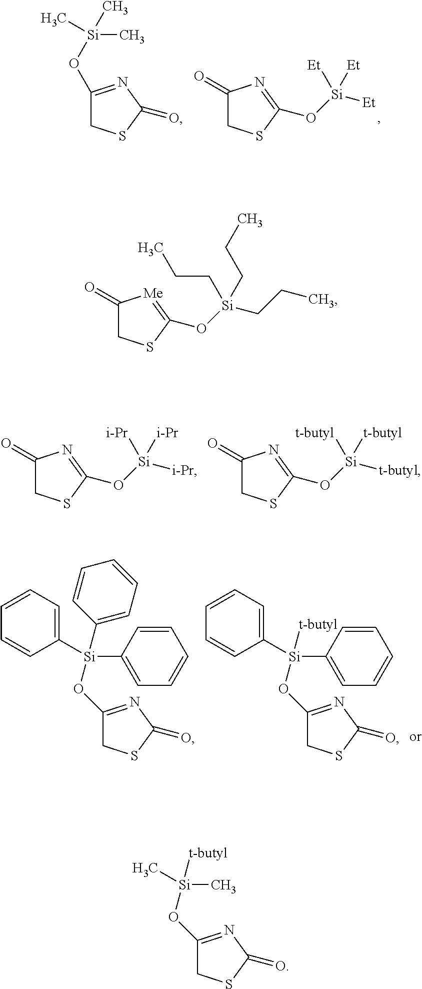

Another aspect of the present invention provides a compound selected from

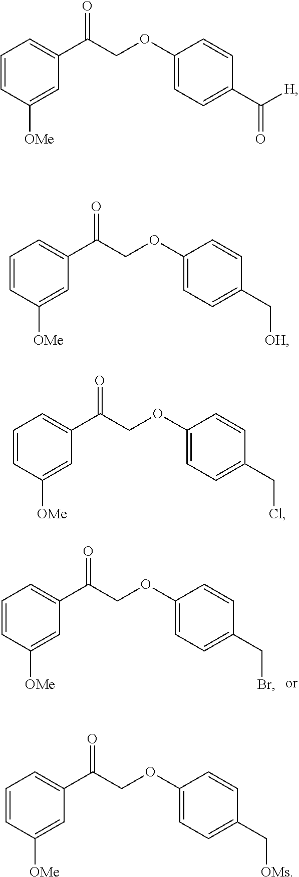

### VI. EXAMPLES

### Example No. 1

### Preparation of 2-[4-(bromomethyl)phenoxy]-1-(3-methoxyphenyl)ethanone

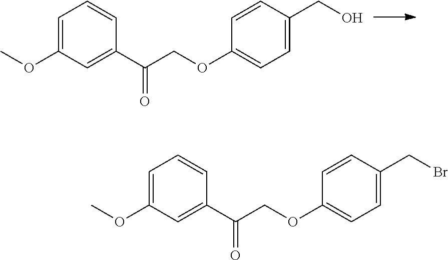 [SMILES: COc1cccc(C(=O)COc2ccc(CO)cc2)c1 ; COc1cccc(C(=O)COc2ccc(CBr)cc2)c1]

A mixture of 2-[4-(hydroxymethyl)phenoxy]-1-(3-methoxyphenyl)ethanone (1.73 g, 6.35 mmol; Supplier: Kalexsyn; Lot=903-TTP-227) in DCM (25 ml) and TIM (5 ml) was added to triphenylphosphine dibromide (2.95 g, 6.99 mmol) portionwise over 10 minutes. The resulting mixture was stirred at room temperature overnight. When TLC indicated that the reaction was complete, the mixture was partitioned between DCM and saturated NaHCO₃, and the aqueous phases were extracted with DCM. The combined organic phases were washed with brine, dried on (Na₂SO₄), filtered, and evaporated in vacuo. The residue was dissolved in DCM and chromatographed on a Biotage column eluting with 25% EtOAc/hex. Fractions containing product were combined and evaporated in vacuo to give 0.94 g of the title compound as a white solid.

| Test | Results |
|---|---|
| Physical Appearance | White solid |
| ¹H NMR Spectrum | δ 7.55 (m, 2H), 7.42 (t, J = 7.88 Hz, 1H), |
| (400 MHz, CDCl₃) | 7.33 (d, J = 8.71 Hz, 2H), 7.18 (dd, J = 7.88, |
| | 2.28 Hz, 1H), 6.91 (d, J = 8.71 Hz), |
| | 5.24 (s, 2H), 4.48 (s, 2H), 3.87 (s, 3H). |
| HPLC Analysis | Not Available |
| Mass Spectrum | Not Available |

### Example 2

### Assays

Assays for Measuring Reduced PPARγ Receptor Activation

Whereas activation of the PPARγ receptor is generally believed to be a selection criteria to select for molecules that may have anti-diabetic and insulin sensitizing pharmacology, this invention finds that activation of this receptor should be a negative selection criterion. Molecules will be chosen from this chemical space because they have reduced, not just selective, activation of PPARγ. The optimal compounds have at least a 10-fold reduced potency as compared to pioglitazone and less than 50% of the full activation produced by rosiglitazone in assays conducted in vitro for transactivation of the PPARγ receptor. The assays are conducted by first evaluation of the direct interactions of the molecules with the ligand binding domain of PPARγ. This can be performed with a commercial interaction kit that measures the direct interaction by florescence using rosiglitazone as a positive control. Further assays can be conducted in a manner similar to that described by Lehmann et al. [Lehmann J M, Moore L B, Smith-Oliver T A: An Antidiabetic Thiazolidinedione is a High Affmity Ligand for Peroxisome Proliferator-activated Receptor (PPAR) J. Biol. Chem.(1995) 270: 12953] but will use luciferase as a reporter as in Vosper et al. [Vosper, H., Khoudoli, G A, Palmer, C N (2003) The peroxisome proliferators activated receptor d is required for the differentiation of THP-1 moncytic cells by phorbol ester. Nuclear Receptor 1:9]. Compound stocks will be dissolved in DMSO and added to the cell cultures at final concentrations of 0.1 to 100 μM and the relative activation will be calculated as induction of the reporter gene (luciferase) as corrected for by the expression of the control plasmid (coding for galactosidase). Pioglitazone and rosiglitazone will be used as reference compounds as described above.

In addition to showing the reduced activation of the PPARγ receptor in vitro, the compounds will not produce significant activation of the receptor in animals. Compounds dosed to full effect for insulin sensitizing actions in vivo (see below) will be not increase activation of PPARγ in the liver as measured by the expression of a P2, a biomarker for ectopic adipogenesis in the liver [Matsusue K, Haluzik M, LambertG, Yim S-H, Oksana Gavrilova O, Ward J M, Brewer B,Reitman M L, Gonzalez F J. (2003) Liver-specific disruption of PPAR in leptin-deficient mice improves fatty liver but aggravates diabetic phenotypes. J. Clin. Invest.; 111: 737] in contrast to pioglitazone and rosiglitazone, which do increase a P2 expression under these conditions.

The insulin sensitizing and antidiabetic pharmacology are measured in the KKAy mice as previously reported [Hofmann, C., Lornez, K., and Colca, J.R. (1991). Glucose transport deficiency corrected by treatment with the oral anti-hyperglycemic agent Pioglitazone. Endocrinology, 129:1915-1925.] Compounds are formulated in 1% sodium carboxy methylcellulose, and 0.01% tween 20 and dosed daily by oral gavage. After 4 days of once daily treatment, treatment blood samples are taken from the retro-orbital sinus and analyzed for glucose, triglycerides, and insulin as described in Hofmann et al. Doses of compounds that produce at least 80% of the maximum lowering of glucose, triglycerides, and insulin will not significantly increase the expression of a P2 in the liver of these mice.

Measuring PPARγ Receptor Activation

The ability of several exemplary compounds of the present invention to bind to PPARγ was measured using a commercial binding assay (Invitrogen Corporation, Carlsbad, Calif.) that measures the test compounds ability to bind with PPAR-LBD/Fluormone PPAR Green complex. These assays were performed on three occasions with each assay using four separate wells (quadruplicate) at each concentration of tested compound. The data are mean and SEM of the values obtained from the three experiments. Rosiglitazone was used as the positive control in each experiment. Compounds were added at the concentrations shown, which range from 0.1-100 micromolar.

Glucose, Insulin, and Triglyceride in Diabetic KKAy Mice Treated with Exemplary Compounds of the Present Invention.

The insulin sensitizing and antidiabetic pharmacology are measured in the KKAy mice as previously reported [Hofmann, C., Lornez, K., and Colca, J.R. (1991). Glucose transport deficiency corrected by treatment with the oral anti-hyperglycemic agent Pioglitazone. Endocrinology, 129:1915-1925.]. Compounds are formulated in 1% sodium carboxy methylcellulose, and 0.01% Tween 20 and dosed daily by oral gavage. After 4 days of once daily treatment, blood samples are taken from the retro-orbital sinus and analyzed for glucose, triglycerides, and insulin as described in Hofmann et al. Doses of compounds that produce at least 80% of the maximum lowering of glucose, triglycerides, and insulin will not significantly increase the expression of a P2 in the liver of these mice.

Compounds were formulated by suspension and orally dosed to KKAy mice at 93 mg/kg for 4 days. The compounds were first dissolved in DMSO and then placed into aqueous suspension containing 7-10% DMSO, 1% sodium methylcarboxycellulose, and 0.01% Tween 20. On the fifth day, the mice were fasted and blood samples were obtained approximately 18 hours after the last dose. The parameters were measured by standard assay methods. Data are mean and SEM N=6-12 mice.

TABLE A
Assay Results

| Cmpd. | | Glucose | Insulin | TG | |
|---|---|---|---|---|---|
| No. | Ex. Description | (Mean/SD) | (Mean/SD) | (Mean/SD) | Glucose |
| 1 | Vehicle A | 518 59 | 24 5 | 284 36 | |
| 2 | 5-[4-(2-oxo-2- phenylethoxy) benzyl]-1,3- thiazolidine-2,4-dione 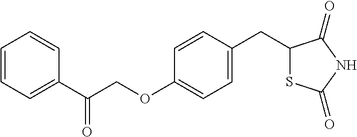 [SMILES: O=C1NC(=O)C(Cc2ccc(OCC(=O)c3ccccc3)cc2)S1] | 0.71 0.03 | 0.13 0.02 | 0.56 0.05 | 36.5 2.4 |
| 3 | 5-{4-[2-(4- fluorophenyl)-2- oxoethoxy]benzyl}- 1,3-thiazolidine-2,4- dione 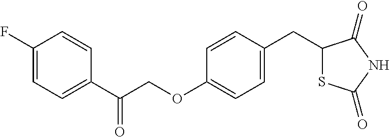 [SMILES: O=C1NC(=O)C(Cc2ccc(OCC(=O)c3ccc(F)cc3)cc2)S1] | 0.61 0.02 | 0.10 0.02 | 0.45 0.02 | 63.7 12.2 |
| 4 | 5-{4-[2-(2- fluorophenyl)-2- oxoethoxy]benzyl}- 1,3-thiazolidine-2,4- dione 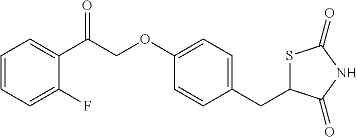 [SMILES: O=C1NC(=O)C(Cc2ccc(OCC(=O)c3ccccc3F)cc2)S1] | 0.64 0.02 | 0.20 0.07 | 0.62 0.04 | 97.9 4.0 |
| 5 | 5-{4-[2-(3- fluorophenyl)-2- oxoethoxy]benzyl)- 1,3-thiazolidine-2,4- dione 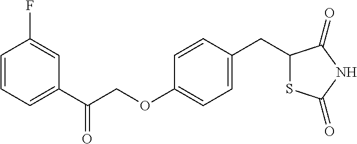 [SMILES: O=C1NC(=O)C(Cc2ccc(OCC(=O)c3cccc(F)c3)cc2)S1] | 0.62 0.05 | 0.24 0.05 | 0.46 0.07 | 64.8 17.5 |
| 6 | 5-{4-[2-(3- methoxyphenyl)-2- oxoethoxy]benzyl}-1,3- thiazolidine-2,4-dione 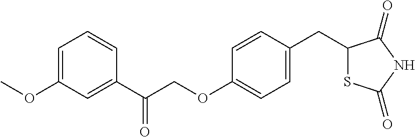 [SMILES: COc1cccc(C(=O)COc2ccc(CC3SC(=O)NC3=O)cc2)c1] | 0.56 0.05 | 0.22 0.03 | 0.41 0.06 | 13.2 0.6 |
| 7 | 5-{4-[2-(2- methoxyphenyl)-2- oxoethoxy]benzyl}- 1,3-thiazolidine-2,4- dione 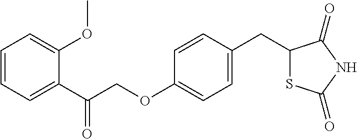 [SMILES: COc1ccccc1C(=O)COc1ccc(CC2SC(=O)NC2=O)cc1] | 0.75 0.04 | 1.20 0.27 | 0.80 0.11 | 76.2 5.6 |
| 8 | 5-{4-[2-(3- chlorophenyl)-2- oxoethoxy]benzyl}- 1,3-thiazolidine-2,4- dione 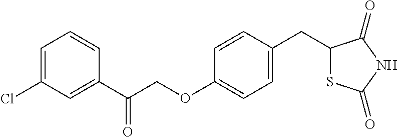 [SMILES: O=C1NC(=O)C(Cc2ccc(OCC(=O)c3cccc(Cl)c3)cc2)S1] | 0.54 0.03 | 0.59 0.33 | 0.43 0.04 | 74.4 1.4 |
| 9 | 5-{4-[2-(2- chlorophenyl)-2- oxoethoxy]benzyl}- 1,3-thiazolidine-2,4- dione 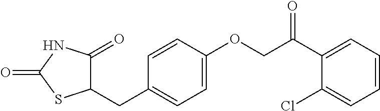 [SMILES: O=C1NC(=O)C(Cc2ccc(OCC(=O)c3ccccc3Cl)cc2)S1] | 1.05 0.03 | 0.47 0.04 | 0.97 0.10 | — |
| 10 | 5-{4-[2-(4- methoxyphenyl)-2- oxoethoxy]benzyl}- 1,3-thiazolidine-2,4- dione 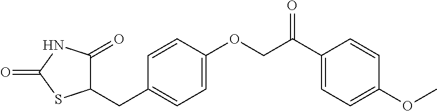 [SMILES: COc1ccc(C(=O)COc2ccc(CC3SC(=O)NC3=O)cc2)cc1] | 1.00 0.03 | 0.76 0.21 | 0.90 0.06 | 37.2 5.0 |

Compound nos. 2-6 exhibited a plasma insulin level of less than about 5 ng/ml and compound no. 7 exhibited a plasma insulin level between about 10 and 20 ng/ml; compound nos. 2-6 exhibited a plasma triglyceride level of between about 100 and 200 mg/dl and compound no. 7 exhibited a plasma triglyceride level between about 300 and 400 mg/dl; and compound nos. 2-6 exhibited a plasma glucose level of between about 350 and 425 mg/dl and compound no. 7 exhibited a plasma glucose level between about 450 and 525 mg/dl.

The PPARγ-sparing compounds of this invention will be more effective for the treatment of diseases caused by metabolic inflammation such as diabetes and metabolic syndrome by limiting the side effects attributable to direct and partial activation of nuclear transcription factors.

Because the compounds of the present invention exhibit reduced PPARγ activation, it is anticipated that these compounds are suitable for use in combination with other compounds having antidiabetic activity, such as metformin, DDP-4 inhibitors, or other antidiabetic agents that function by differing mechanisms to augment the actions or secretions of GLP1 or insulin. Specifically because of the reduced PPARγ interaction, these compounds will also be useful for treating dyslipidemia associated with metabolic inflammatory diseases combining particularly well with lipid lowering statins such as atorvastatin or the like. It is also anticipated that the combination of a compound of Formula I and other antidiabetic compounds will be more effective in treating diabetes than combinations with PPAR-activating compounds as they will avoid side effects associated with PPARγ activation that may include volume expansion, edema, and bone loss.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A process of for preparing a compound of Formula I: 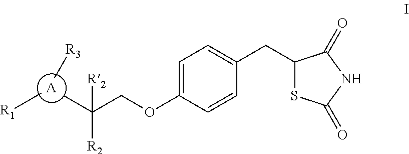
or a pharmaceutically acceptable salt thereof, wherein
Each of R₁ and R₃ is independently selected from H, halo, aliphatic, and alkoxy, wherein the aliphatic or alkoxy is optionally substituted with 1-3 of halo;
Each of R′₂ and R₂ are independently selected from —H, halo, hydroxy, or optionally substituted aliphatic, alkoxy, —O-acyl, —O-aroyl, —O-heteroaroyl, —O(SO₂)NH₂, —O—CH(Rₘ)OC(O)Rₙ, —O—CH(Rₘ)OP(O)(ORₙ)₂ —O—P(O)(ORₙ)₂, or 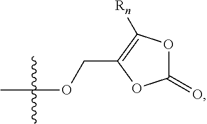
wherein each Rₘ is independently C₁₋₆ alkyl, each Rₙ is independently C₁₋₁₂ alkyl, C₃₋₈ cycloalkyl, or phenyl, each of which is optionally substituted; or
R₂ and R′₂ together form oxo; or
R₂ and R′₂ together form —O(CH₂)ₙO—, wherein n is 2 or 3; or
R₂ and R′₂ together form —S(CH₂)ₘS—, wherein m is 2 or 3; and
Ring A is phenyl, pyridin-2-yl, pyridin-3-yl or pyridin-4-yl, each of which is substituted with R₁ and R₃ groups;
comprising the step of:
reacting a compound of Formula 2A: 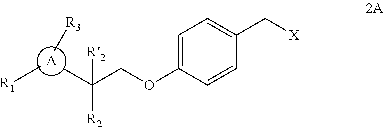
wherein X is a leaving group, with a compound of Formula 3A 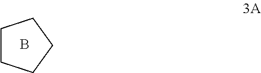
wherein ring B of Formula 3A is selected from 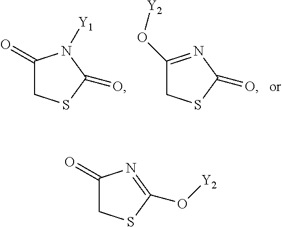
wherein Y₁ is hydrogen or PG_{N} and Y₂ is PG_{O}, wherein PG_{N} is a nitrogen protecting group and PG_{O} is an oxygen protecting group, to form a compound of Formula 4A; and 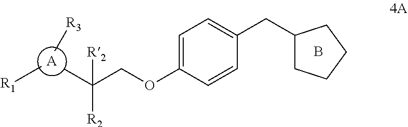
when Y₁ is PG_{N} or when Y₂ is present, deprotecting the compound of Formula 4A to form a compound of Formula I.

2. The process of claim 1, wherein X is a leaving group selected from —Br, —Cl, —I,—OMs, —OTs, —OTf, —OBs, —ONs, —O-tresylate, or —OPO(OR₄)₂, wherein each R₄ is independently C₁₋₄ alkyl or two of R₄ together with the oxygen and phosphorous atoms to which they are attached form a 5-7 membered ring.

3. The process of claim 1, further comprising modifying a compound of Formula 5A 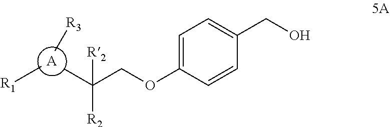
to form a compound of Formula 2A.

4. The process of claim 3, further comprising reacting a compound of Formula 6A 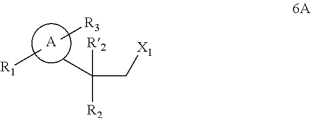
wherein X₁ is halo, with a compound having the structure 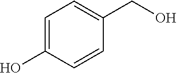
under basic conditions to form a compound of Formula 5A.

5. The process of claim 4, wherein the compound of Formula 6A comprises

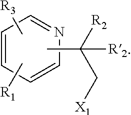

6. The process of claim 5, wherein the compound of Formula 6A comprises

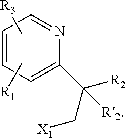

7. The process of claim 6, wherein the compound of Formula 6A comprises 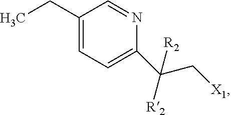
wherein X₁ is —Br or —Cl.

8. The process of claim 7, wherein R₂ and R′₂ together form oxo.

9. The process of claim 4, wherein the compound of Formula 6A comprises

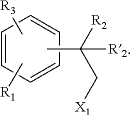

10. The process of claim 9, wherein the compound of Formula 6A comprises 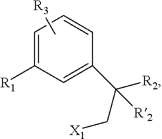
wherein R₁ is a C₁₋₆ alkyl or C₁₋₆ alkoxy, either of which is optionally substituted with 1-3 halo.

11. The process of claim 10, wherein the compound of Formula 6A comprises

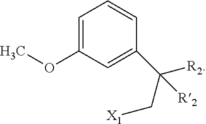

12. The process of claim 11, wherein R₂ and R′₂ together form oxo.

13. The process of claim 1, wherein X and X₁ are independently selected from —Br and —Cl.

14. The process of claim 1, wherein ring B of Formula 3A is 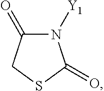
Y₁ is PG_{N}, and PG_{N} is a nitrogen protecting group selected from Cbz, Moz, Boc, Fmoc, Ac, Bz, Bn, PMB, DMPM, PMP, or trityl.

15. The process of claim 1, wherein ring B of Formula 3A is 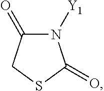
and Y, is hydrogen.

16. The process of claim 1, wherein ring B of Formula 3A is 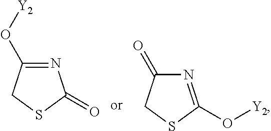
Y₂ is PG_{O}, and PG_{O} is an oxygen protecting group selected from —Si(R₆)₃, optionally substituted alkyl, or optionally substituted alkylcarbonyl, wherein each R₆ is independently straight or branched C₁₋₄ alkyl.

17. The process of claim 16, wherein ring B of Formula 3A is 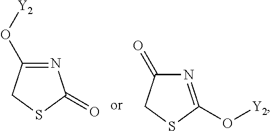
Y₂ is PG_{O}, and PG_{O} is —Si(R₆)₃, wherein each R₆ is independently selected from methyl, ethyl, propyl, isopropyl, butyl, sec-butyl, or tert-butyl.

18. The process of claim 16, wherein ring B of Formula 3A is 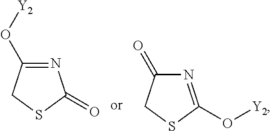
Y₂ is PG_{O}, and PG_{O} is a C₁₋₆ alkyl or a C₁₋₆ alkylcarbonyl.

19. A compound selected from

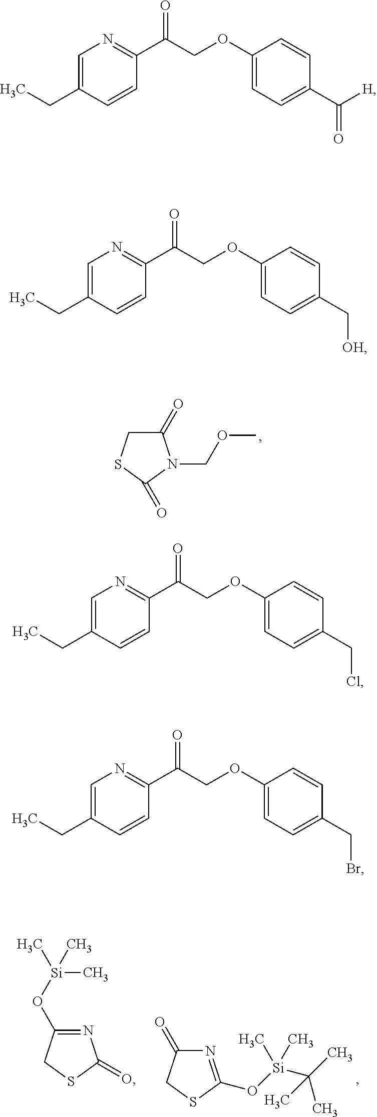

20. A compound selected from [chemical structure]
